(19) United States
(12) Patent Application Publication
(10) Pub. No.: US 2012/0241470 A1
(43) Pub. Date: Sep. 27, 2012

(54) Sanitization Dispenser Systems

(75) Inventors: David Snodgrass, Jupiter, FL (US); Jeffrey J. Rospierski, Alden, NY (US)

(73) Assignee: ULTRACLENZ, LLC, Jupiter, FL (US)

(21) Appl. No.: 13/430,476

(22) Filed: Mar. 26, 2012

Related U.S. Application Data
(60) Provisional application No. 61/467,676, filed on Mar. 25, 2011.

(51) Int. Cl. A47K 5/12; B67D 7/08

(52) U.S. Cl. 222/63; 222/639; 222/52; 222/504

(57) ABSTRACT

Various arrangements for controlling the dispensing of hand sanitizer dispensed from a hand hygiene dispenser provide for a user to adjust the dosage of a product dispensed, including electronic control and mechanical control.

### CROSS REFERENCE TO RELATED APPLICATIONS

This application claims priority to U.S. Application Ser. No. 61/467,676 filed Mar. 25, 2011, which is incorporated by reference herein. This application also incorporates by reference U.S. patent Ser. No. 11/146,955 filed Jun. 7, 2005; Ser. No. 11/146,828 filed Jun. 7, 2005; Ser. No. 11/804,675 filed May 18, 2007; Ser. No. 12/150,223 filed Apr. 25, 2008; Ser. No. 12/480,285 filed Jun. 8, 2009; Ser. No. 12/560,250 filed Sep. 15, 2009; Ser. No. 13/215,823 filed Aug. 23, 2011; and 61/486,491 filed May 16, 2011, and U.S. Pat. Nos. 8,056,768; 8,020,733; 7,104,519; 6,431,400; 6,426,701; and 6,347,724.

### BACKGROUND OF THE INVENTION

The present invention relates to sanitization dispenser systems, and in particular to a field adjustable dose volume dispenser, power circuit which improves battery life, and manual and automatic actuator mechanisms.

One dispensing system currently available through UltraClenz, called the ProClenz dispenser, has an actuate piston pump with a fixed diameter. The dose volume output is determined by the length of the stroke or travel of the piston within the cylinder. To adjust the dose volume output, the stroke length needs to be adjusted. One aspect of the present invention addresses this disadvantage. Another aspect of the invention relates to prolonging battery life in a dispenser. Another aspect of the invention relates to manual and automatic actuator mechanisms.

### SUMMARY OF THE INVENTION

The present invention provides a dispenser for dispensing sanitizer solution for hand hygiene, comprising: a valve having an open position for dispensing sanitizer solution, and a closed position for preventing dispensing of sanitizer solution; a sensor for sensing the presence of a user's hands; and a control circuit responsive to the sensor for controlling the time that the valve is open and thus control the amount of solution dispensed.

The invention provides a dispenser for dispensing sanitizer solution for hand hygiene, comprising: a valve having an open position for dispersing sanitizer solution, and a closed position for preventing dispensing of sanitizer solution, said valve comprising a dose adjustable index block, said index block having a ramp with a plurality of ramp seats at different axial positions along the index block, and a dose ramp pin which can be moved between the ramp seats, and wherein the amount of sanitizer solution dispensed is dependent on the ramp seat in which the ramp pin is seated.

The invention provides a dispenser for dispersing sanitizer solution for hand hygiene comprising: a valve having an open position for dispensing sanitizer solution, and a closed position for preventing dispensing of sanitizer solution, said valve comprising a piston slideable within a cylinder to the open and closed positions; and a push bar for actuation by a user, said push bar pivoting on a pivot point, said push bar raising a helix activation mechanism which slides the piston to open the valve in response, to dispense sanitizer solution.

The invention provides a dispenser for dispensing sanitization solution for hand hygiene, comprising: a valve having an open position for dispensing sanitizer solution, and a closed position for preventing dispensing of sanitizer solution, said valve comprising a piston slideable within a cylinder to the open and closed positions; a sensor for sensing the presence of a user's hands; a motor for opening the valve; and a motor controller for energizing the motor to raise an activation mechanism to open the valve in response to the sensor sensing a user's hands.

### BRIEF DESCRIPTION OF THE DRAWING

FIG. 1 shows four D-cell batteries connected in a series arrangement;

FIG. 2 shows two sets of D-cell batteries connected in a parallel arrangement;

FIG. 3 shows a basic schematic of a boost converter;

FIG. 4 shows one embodiment of a dose adjustable index block;

FIG. 5 shows an arrangement for a manual link arm lift mechanism, wherein linkage members are used as mechanical advantage to raise and lower a pump block;

FIG. 6 shows a dual gear/cam push lift mechanism wherein an opposing gear mesh drives both gears at the same time;

FIG. 7 shows a manual cam scissor lift mechanism;

FIG. 8 shows a planetary cam scissor lift arrangement similar to FIG. 7;

FIG. 9 shows a manual helical lift mechanism;

FIG. 10 shows a helical lift mechanism similar to that of FIG. 9, but with a DC motor;

FIG. 11 shows a manual drive gear platform mechanism;

FIG. 12 shows an automotive direct drive gear platform mechanism with a DC motor;

FIG. 13 shows a block diagram of a controller; and

FIG. 14 shows a cross-sectional elevation view of a dispenser.

### DESCRIPTION OF THE PREFERRED EMBODIMENTS OF INVENTION

Various preferred embodiments will be described, but the present invention is not limited to theses embodiments.

The present invention provides a dispenser for dispensing sanitizer solution for hand hygiene, comprising: a valve having an open position for dispensing sanitizer solution, and a closed position for preventing dispensing of sanitizer solution; a sensor for sensing the presence of a user's hands; and a control circuit responsive to the sensor for controlling the time that the valve is open and thus control the amount of solution dispensed.

The dispenser may further include a timer for setting the amount of time, wherein the timer is adjustable. The control circuit may comprise a microcontroller, a motor controlled by the microcontroller, said motor controlling the time that the valve is open. The dispenser may further include a cam driven by the motor which drives a lever arm which opens the valve. The dispenser may further include a circumferential cam having a plurality of ribs arranged circumferentially around the cam, wherein rotation of the cam causes the ribs to sequentially engage a switch, the number of ribs engaging the switch determining the amount of time that the valve is open and the amount of solution dispersed. The control circuit may include a cam driven by a motor which controls opening of the valve by an optical transmitter and optical sensor, the cam defining openings through which light from the optical transmitter passes to reach the optical sensor upon rotation of the cam, the optical sensor activating the opening of the valve when light is received from the optical transmitter through the openings. The control circuit may include a cam driven by a motor which controls opening of the valve by at least one magnet mounted on the cam, and at least one magnetic sensor which detects the magnet when the cam rotates, the magnetic sensor activating the opening of the valve when the magnetic sensor detects the magnet. The control circuit may comprise a stepper motor which opens the valve only when the motor is stepped. The stepper motor may be stepped a plurality of cycle times for each dispensing. The control circuit may include a D.C. motor for controlling the time that the valve is open, said motor being energized by a D.C. source and a step-up converter which outputs a D.C. voltage greater than the voltage of the D.C. source. The D.C. source may comprise batteries connected in parallel.

The invention provides a dispenser for dispensing sanitizer solution for hand hygiene, comprising: a valve having an open position for dispersing sanitizer solution, and a closed position for preventing dispensing of sanitizer solution, said valve comprising a dose adjustable index block, said index block having a ramp with a plurality of ramp seats at different axial positions along the index block, and a dose ramp pin which can be moved between the ramp seats, and wherein the amount of sanitizer solution dispensed is dependent on the ramp seat in which the ramp pin is seated.

The invention provides a dispenser for dispersing sanitizer solution for hand hygiene comprising: a valve having an open position for dispensing sanitizer solution, and a closed position for preventing dispensing of sanitizer solution, said valve comprising a piston slideable within a cylinder to the open and closed positions; and a push bar for actuation by a user, said push bar pivoting on a pivot point, said push bar raising a helix activation mechanism which slides the piston to open the valve in response, to dispense sanitizer solution.

The cylinder may have a valve seat at one end, and the piston may rest on the valve seat to close the valve, and wherein the valve is open when the piston is displaced from the valve seat.

The invention provides a dispenser for dispensing sanitization solution for hand hygiene, comprising: a valve having an open position for dispensing sanitizer solution, and a closed position for preventing dispensing of sanitizer solution, said valve comprising a piston slideable within a cylinder to the open and closed positions; a sensor for sensing the presence of a user's hands; a motor for opening the valve; and a motor controller for energizing the motor to raise an activation mechanism to open the valve in response to the sensor sensing a user's hands.

The cylinder may have a valve seat at one end, and the piston may rest on the valve seat to close the valve, and wherein the valve is open when the piston is displaced from the valve seat.

The present invention provides for adjusting the dose of product dispensed by a hand hygiene sanitizer dispenser. A mechanical means of adjustment is one way to adjust the dose volume and it would be applicable to both manual and touch-free (also called “automatic”) dispensers. However, it may be difficult to create a mechanical design that is cost effective, utilizes minimal internal gearbox volume and that is easy to execute adjustment in the field by an end user. As an alternative, several electronic means are available. However, they will only apply to the touch-free version of the dispenser.

The current touch-free dispenser has a fixed stroke length that is driven electronically by a micro controller (μC) that in turn drives a DC motor attached to a series of gears that drive a cam which drives a lever arm that contacts the pump to push the pump's piston into its cylinder and thus displace chemical into the user's hand. The cam is indexed by a micro switch that is attached electrically to the μC and provides feedback allowing the μC to determine the cam's correct starting position as well as when the cam has completed a complete revolution.

If 180° of cam revolution produces the maximum linear travel of the lever arm corresponding to the maximum stroke of the pump's piston, then reducing the pump's dose volume output can be accomplished by limiting the cam's radial motion to less than 180° of revolution.

Following are descriptions of various embodiments that could be implemented electronically to control the radial motion of the cam.

### Embodiment 1

### Timing Control

The μC can be programmed to allow the motor to run for a predetermined period of time. This time will correspond to a predictable amount of cam radial motion that is less than 180° of revolution which results in a predictable dose volume that is less than the maximum dose volume. The μC will start the motor running forward and then start a timer when the cam has cleared the indexing micro switch. At this point, a sanitizing chemical is beginning to dispense. Once the timer has expired, the μC will stop the motor. At this point, the cam will have reached its maximum rotation and chemical will no longer be dispensed. The μC will then reverse the motor and let it run until the cam reaches the indexing micro switch. Once the micro switch is activated, the μC will stop the motor which orients the cam at the correct starting position for the next activation.

The dose volume can be adjusted by changing the amount of time the motor is allowed to run forward. Several preset dose volumes could be programmed into the μC's firmware and selected by a user via switches, buttons or jumpers located on the dispenser. If a particular customer needs custom dose volumes, this can be implemented solely by modifying the firmware. No hardware (mechanical or electronic) modifications are required.

### Embodiment 2

### Micro Switch Control

Ribs can be located around the circumference of the cam at predetermined locations. These ribs will engage the indexing micro switch and correspond to a predictable amount of cam radial motion that is less than 180° of revolution which results in a predictable dose volume that is less than the maximum dose volume. The μC will start the motor running forward. When the cam has cleared the indexing micro switch, the μC will wait for the next indexing micro switch activation which corresponds to a rib on the cam. At this point, chemical is beginning to dispense. If multiple dose volumes are available there will be multiple ribs on the cam. The μC will count the number of micro switch activations until the correct dose volume is reached. Chemical will be dispensed during this time. When the correct micro switch activation count is reached, the μC will stop the motor. At this point, the cam will have reached its maximum rotation and chemical will no longer be dispensed. The μC will then reverse the motor and let it run while counting backwards the micro switch activations until reaching the starting position. The μC will then stop the motor which orients the cam at the correct starting position for the next activation.

The preset dose volumes can be selected by a user via switches, buttons or jumpers located on the dispenser. If a particular customer needs custom dose volumes, a new cam can be manufactured with ribs located at the correct positions.

### Embodiment 3

### Optical Control

The indexing micro switch of embodiments 1 and 2 can be replaced with an optical transmitter (light source i.e. LED) and an optical sensor. The optical transmitter would be placed on one side of the cam near its circumference and the optical sensor would be placed on the opposite side of the cam. The ribs located around the circumference of the cam in embodiment 2 can be replaced with openings molded into the cam near its circumference. When an opening on the cam passes between the optical transmitter and optical sensor, light from the transmitter will pass through the opening and activate the sensor. The sensor activation will be detected by the μC and processed in the same way as the micro switch activation of embodiment 2. The same operational logic of embodiment 2 will then apply.

The preset dose volumes can be selected by a user via switches, buttons or jumpers located on the dispenser. If a particular customer needs custom dose volumes, a new cam could be manufactured with openings located at the correct positions.

### Embodiment 4

### Magnetic Control

The indexing micro switch of embodiments 1 and 2 can be replaced with a magnetic sensor (Hall-Effect sensor) located next to the cam near its circumference. The ribs located around the circumference of the cam in embodiment 2 can be replaced with small magnets embedded into the cam near its circumference. When a magnet on the cam passes near the magnetic sensor, the sensor will activate. The sensor activation will be detected by the μC and processed in the same way as the micro switch activation of embodiment 2. The same operational logic of embodiment 2 will then apply.

The preset dose volumes can be selected by a user via switches, buttons or jumpers located on the dispenser. If a particular customer needs custom dose volumes, a new cam could be manufactured with embedded magnets located at the correct positions.

### Embodiment 5

### Stepper Motor Control

A stepper motor operates by receiving a train of pulses from a driver circuit (H-bridge) that is controlled by the μC. Each pulse will cause the motor to rotate by a precise amount called a step. Each step may be as small as 1° of rotation or less and the number of pulses received will determine the number of steps. A potential problem with stepper motors is that there is no feedback to the μC to make sure that the motor has actually rotated the correct number of steps. To remedy this, an encoder could be used. Many stepper motors can be purchased with an optional built-in encoder.

The μC can be programmed to allow the stepper motor to run for a predetermined number of steps. This will correspond to a precise amount of cam radial motion that is less than 180° of revolution which results in a predictable dose volume that is less than the maximum dose volume. The μC will setup the driver circuit for forward motor rotation and then send pulses to start the motor running forward while keeping a count of each pulse. At this point, chemical is beginning to dispense. Once the desired number of pulses has been sent to the stepper motor, the μC will stop sending pulses and the motor will stop. At this point, the cam will have reached its maximum rotation and chemical will no longer be dispensed. The μC will then reverse the motor and let it run until the cam reaches the indexing micro switch. Once the micro switch is activated, the μC will stop the motor which orients the cam at the correct starting position for the next activation.

The dose volume can be adjusted by changing the number of steps the motor is allowed to run forward. Several preset dose volumes could be programmed into the μC's firmware and selected by a user via switches, buttons or jumpers located on the dispenser. If a particular customer needs custom dose volumes, this can be implemented solely by modifying the firmware. No hardware (mechanical or electronic) modifications are required.

### Dispenser Battery Life Improvement

The UltraClenz ProClenz model touch-free (TF) dispenser uses 4 D-cell alkaline batteries in a series configuration and is able to activate about 36,000 times, at 100 activations per day, for a battery life of about 1 year.

The current capacity of a typical 1.5V D-cell alkaline battery is rated between 12,000 mAh and 20,000 mAh down to 0.8V. By placing the batteries in a series configuration, 4 batteries provides a total voltage of 6.0V but the total current capacity remains the same as 1 battery at 12,000 mAh to 20,000 mAh. The batteries' combined voltage of 6.0V is regulated down using a low-drop out (LDO) linear voltage regulator to 3.3V to power the micro controller (μC) and its associated circuitry. The motor is driven directly from the batteries but the voltage is pulse width modulated (PWM) to produce an average voltage of about 4.0V. The batteries are considered dead, and thus unusable and in need of replacement, when their combined voltage drops to 4.8V. However, the batteries should not be considered dead and in need of replacement until just above 3.3V (the voltage of the regulator) but when the motor is running under a load and the batteries are below 4.8V, the in-rush current required by the motor can momentarily drop the voltage below 2.7V which resets the μC.

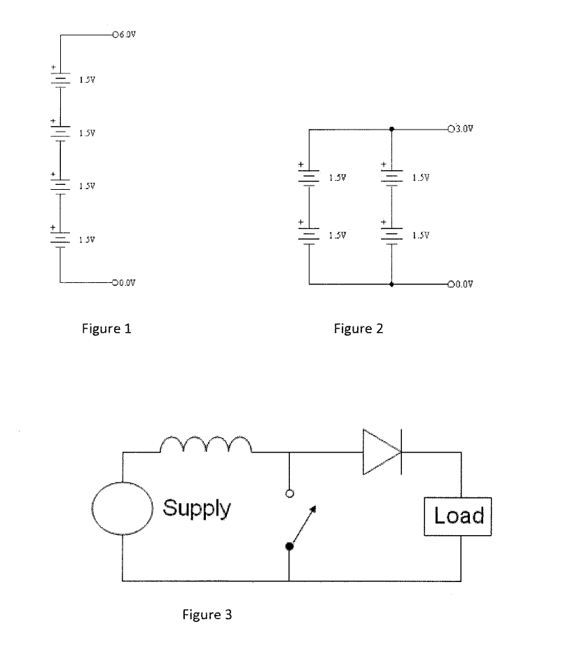

Assume that a set of 4 D-cell alkaline batteries are connected in a series arrangement (see FIG. 1) and have a current capacity of 15,000 mAh or 15 Ah. The total power available with a combined voltage range of 6.0V to 3.2V or 1.5V to 0.8V per cell is Pₜₒₜₐₗ=Vₜₒₜₐₗ×Iₜₒₜₐₗ. Solving for Pₜₒₜₐₗ, we get Pₜₒₜₐₗ=60V×15 Ah, and Pₜₒₜₐₗ=90 Wh.

The voltage cutoff requirement of 4.8V will limit the available power to:

$${P}_{available} = \{ 1 - ( \frac{{v}_{max} - {v}_{cutoff}}{{v}_{max} - {v}_{min}} ) \} \% \times {P}_{total}$$

$${P}_{available} = \{ 1 - ( \frac{6.0 V - 4.8 V}{6.0 V - 3.2 V} ) \} \% \times 90 Wh$$

$${P}_{available} = 57.1 \% \times 90 Wh$$

$${P}_{available} = 51.4 Wh$$

To increase the number of activations per set of batteries a new battery arrangement and a different regulator to power the μC and associated circuitry was considered. The regulator will be of the “boosting” type with 90% efficiency and will be able to supply a regulated 3.0V output with an input from 3.0V down to 0.7V.

If a first set of 2 D-cell alkaline batteries are connected in a series arrangement, the combined voltage is 3.0V and the total current capacity is 12 Ah to 20 Ah. If a second set of D-cell alkaline batteries, also in a series arrangement are connected in a parallel arrangement with the first set (see FIG. 2), the combined voltage is still 3.0V but the combined current capacity doubles becoming 24 Ah to 40 Ah.

Assume that the battery arrangement of FIG. 2 is used and has a combined current capacity of 30 Ah. The total power available with a combined voltage range of 3.0V to 1.6V or 1.5V to 0.8V per cell is Pₜₒₜₐₗ=Vₜₒₜₐₗ×Iₜₒₜₐₗ.

Solving for Pₜₒₜₐₗ, we get Pₜₒₜₐₗ=3.0V×30 Ah, and Pₜₒₜₐₗ=90 Wh.

While the series arrangement and series/parallel arrangement produce the same 90 Wh of total power, because a boosting regulator is being used there is no need for a premature cutoff voltage. The 4.8V cutoff of the series battery arrangement reduced the total available power to 51.4 Wh. Thus, the battery arrangement of FIG. 2 combined with a boosting regulator will provide a 42.8% improvement in battery capacity. This translates to approximately 51,000 activations at 100 activations per day for a battery life of 1.4 years.

A boost converter (also known as a step-up converter) is a power converter with an output DC voltage greater than its input DC voltage. It is a class of switching-mode power supply (SMPS) containing at least two semiconductor switches (a diode and a transistor) and at least one energy storage element. Filters made of capacitors (sometimes in combination with inductors) may be added to the output of the converter to reduce output voltage ripple.

FIG. 3 shows a basic schematic of a boost converter. The switch is typically a MOSFET, IGBT, or BJT.

A boost converter can be characterized as a DC to DC converter with an output voltage greater than the source voltage. A boost converter is sometimes called a step-up converter since it “steps up” the source voltage. Since power (P=VI) must be conserved, the output current is lower than the source current.

A boost converter is used as the voltage increase mechanism in the circuit known as the ‘Joule thief’. This circuit topology is used with low power battery applications, and is aimed at the ability of a boost converter to ‘steal’ the remaining energy in a battery. This energy would otherwise be wasted since the low voltage of a nearly depleted battery makes it unusable for a normal load.

This energy would otherwise remain untapped because there would not be enough current to flow through a load when voltage decreases. This voltage decrease occurs as batteries become depleted, and is a characteristic of alkaline batteries. Since (P=V²/R) as well, and R tends to be stable, power available to the load goes down significantly as voltage decreases.

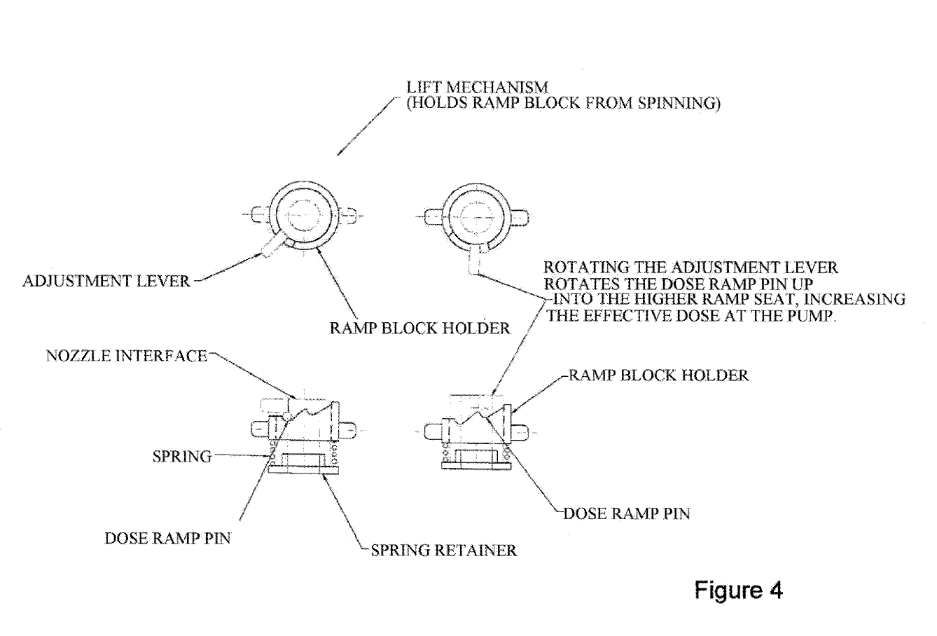

FIG. 4 shows one example of a dose adjustable index block. The amount of the dose can be increased by rotating the adjustment lever which will rotate the dose ramp pin up into a higher ramp sent, as shown in the two figures on the right in FIG. 4.

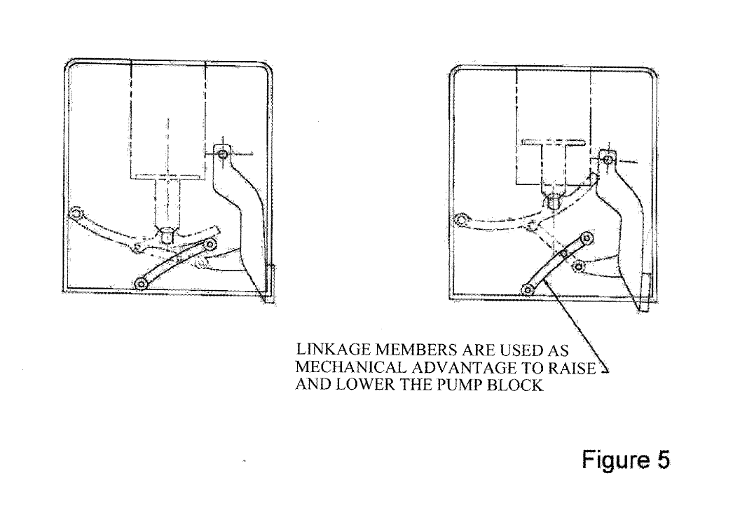

FIGS. 5-12 show various arrangements for manual and automatic dispensers.

FIG. 5 shown a manual link arm lift mechanism, wherein linkage members are used as mechanical advantage to raise and lower a pump block.

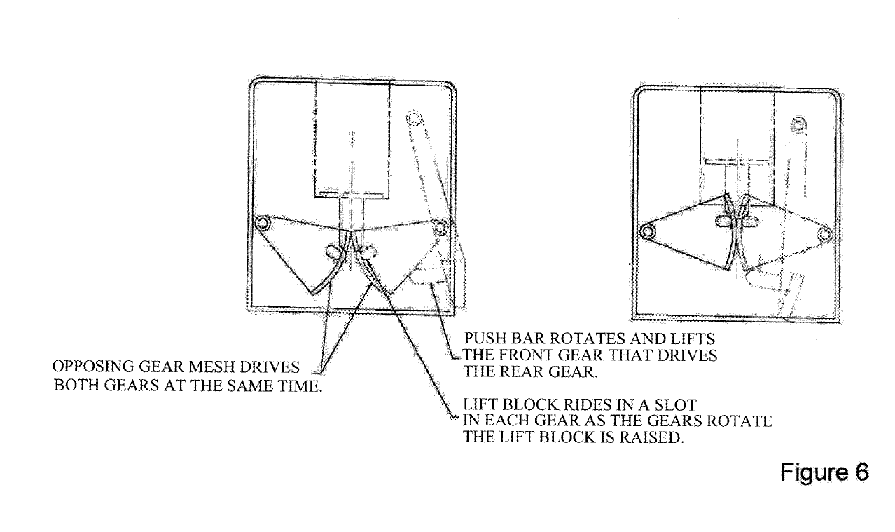

FIG. 6 shows a dual gear/cam push lift mechanism wherein an opposing gear mesh drives both gears at the same time. The push bar rotates and lifts the front gear, which drives the rear gear. The lift block rides in a slot in each gear. As the gears rotate, the lift block is raised.

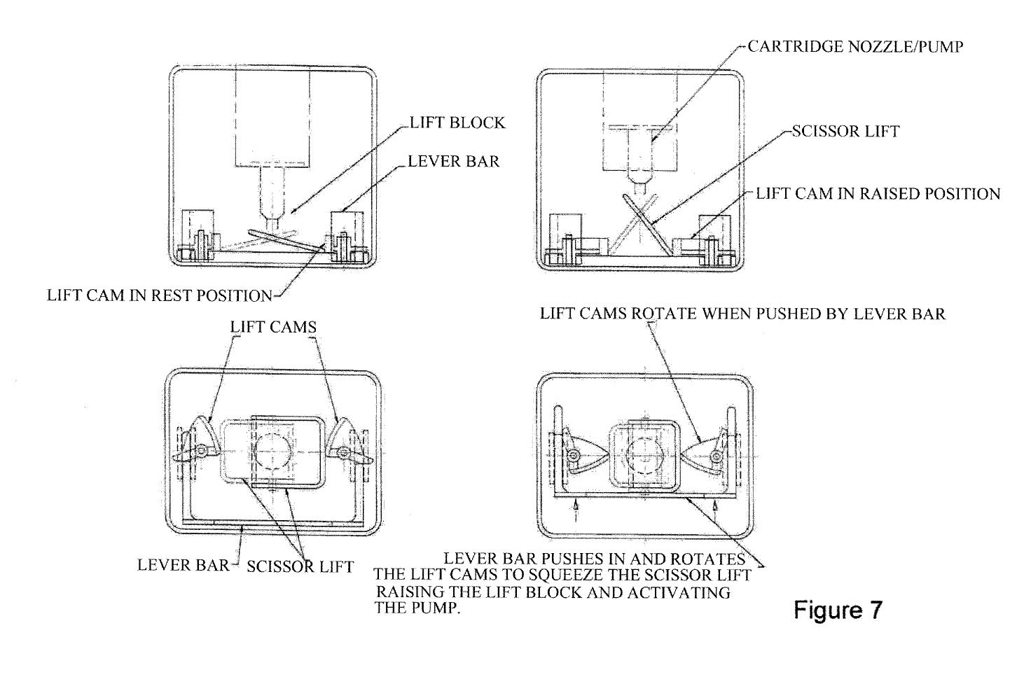

FIG. 7 shows a manual cam scissor lift mechanism. The upper left and lower left show the lift cam in a rest position when a user pushes a lever bar, the lever bar pushes in and rotates the lift cams to squeeze the scissor lift, raising the lift block and activating the pump, as shown in the upper and lower right.

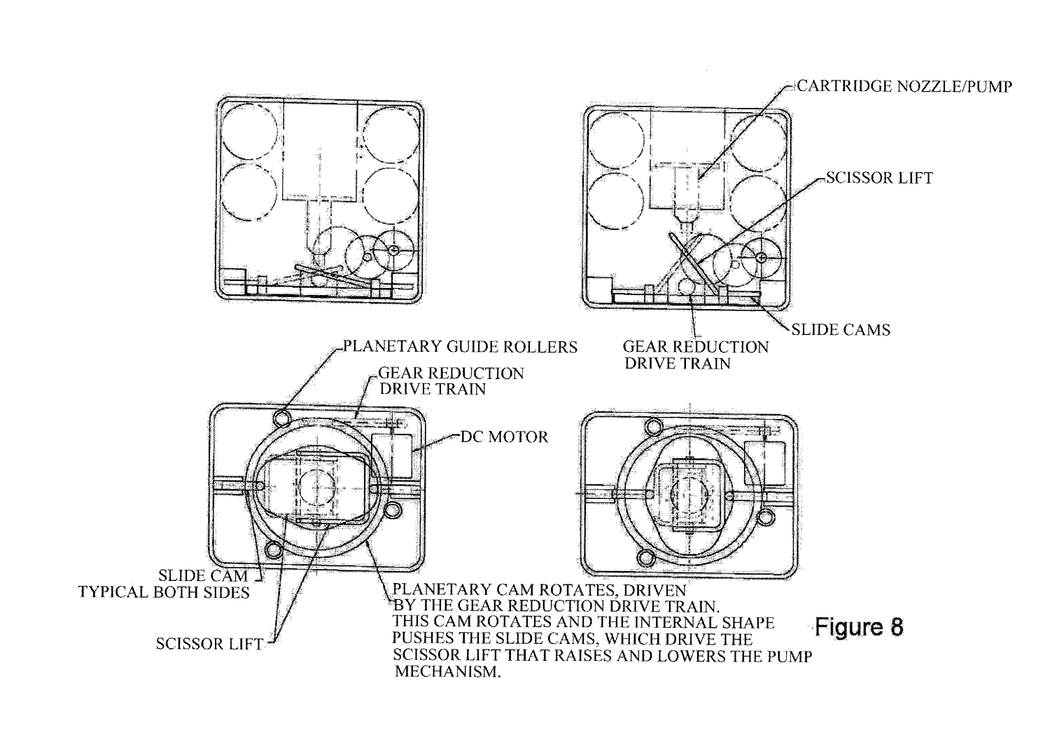

FIG. 8 shows a planetary cam scissor lift similar to FIG. 7. Here a DC motor is provided with a gear reduction drive train. As the cam rotates, its internal surface pushes the slide cams, which drive the scissor lift, which raises and lowers the pump mechanism.

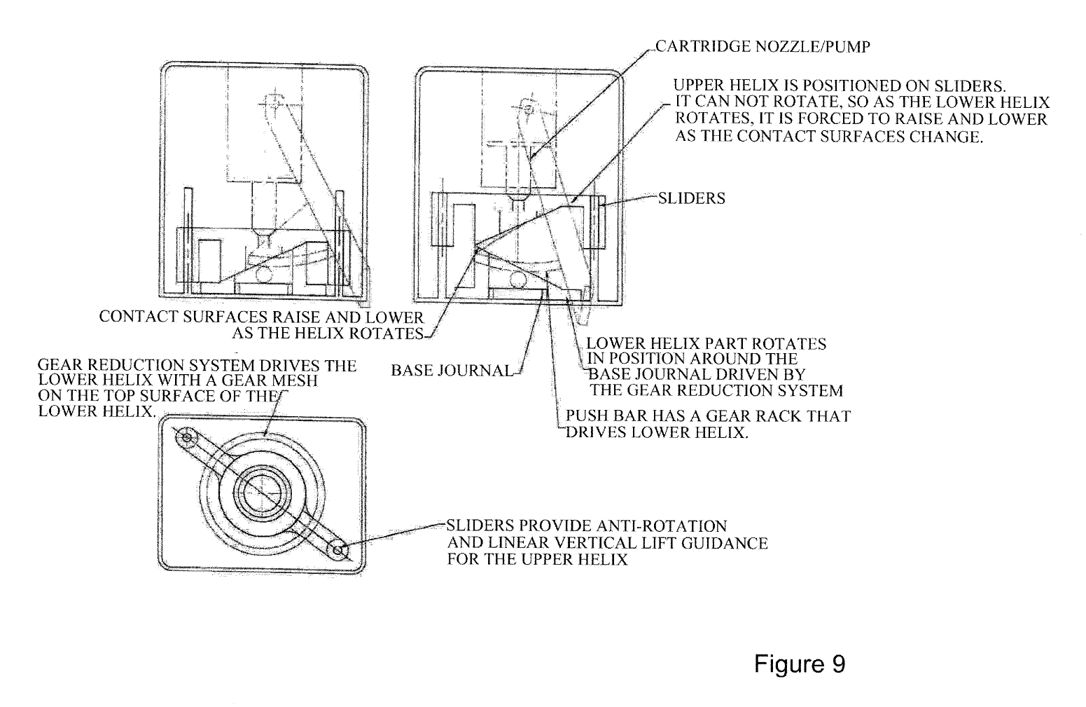

FIG. 9 shows a manual helical lift mechanism. Here the push bar has a gear rack that drives a lower helix. The lower helix part rotates in position around the base journal, driven by the gear reduction system. The upper helix is positioned on sliders and cannot rotate. As the lower helix rotates, it is forced to raise and lower as the contact surfaces change. This will cause the cartridge nozzle/pump to dispense.

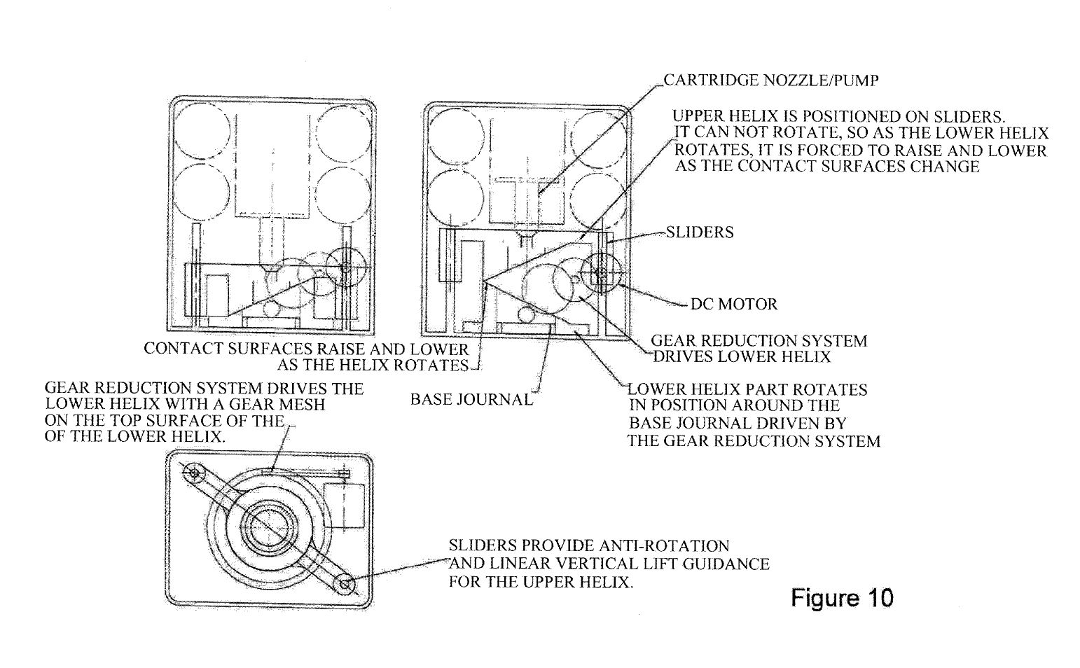

FIG. 10 shows a helical lift mechanism similar to that of FIG. 9, but with a DC motor.

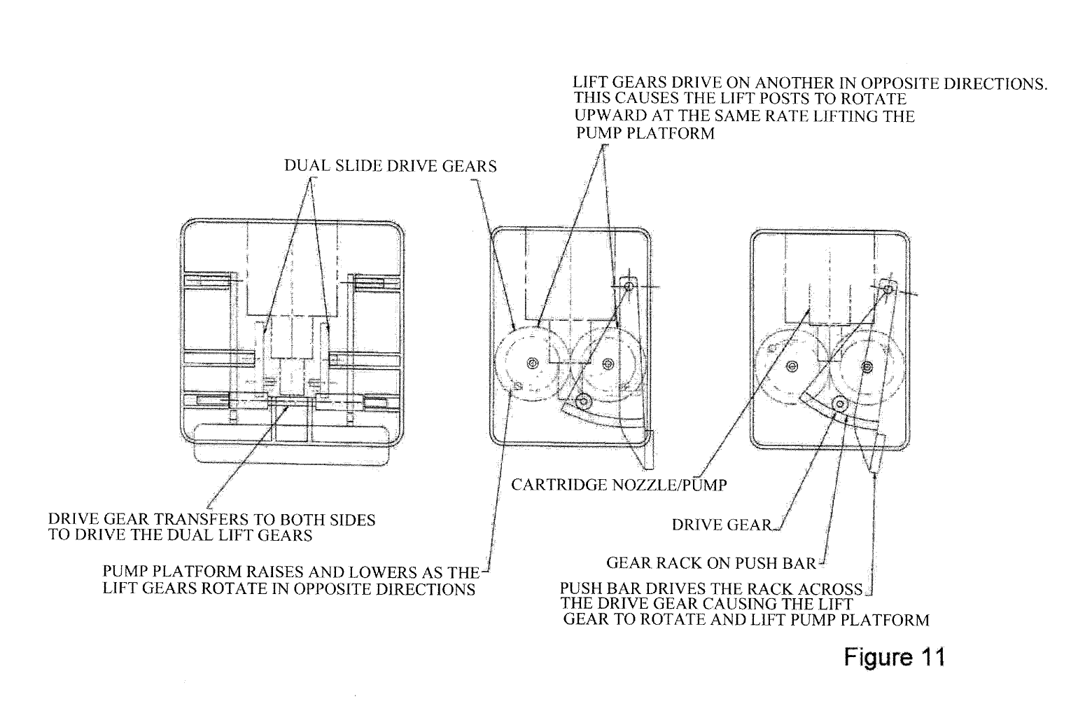

FIG. 11 shows a manual drive gear platform mechanism. Here the push bar drives the rack across the drive gear, causing the lift gear to rotate and lift the pump platform. The lift gears drove one another in opposite directions. This causes the lift posts to rotate upward at the same rate, lifting the pump platform.

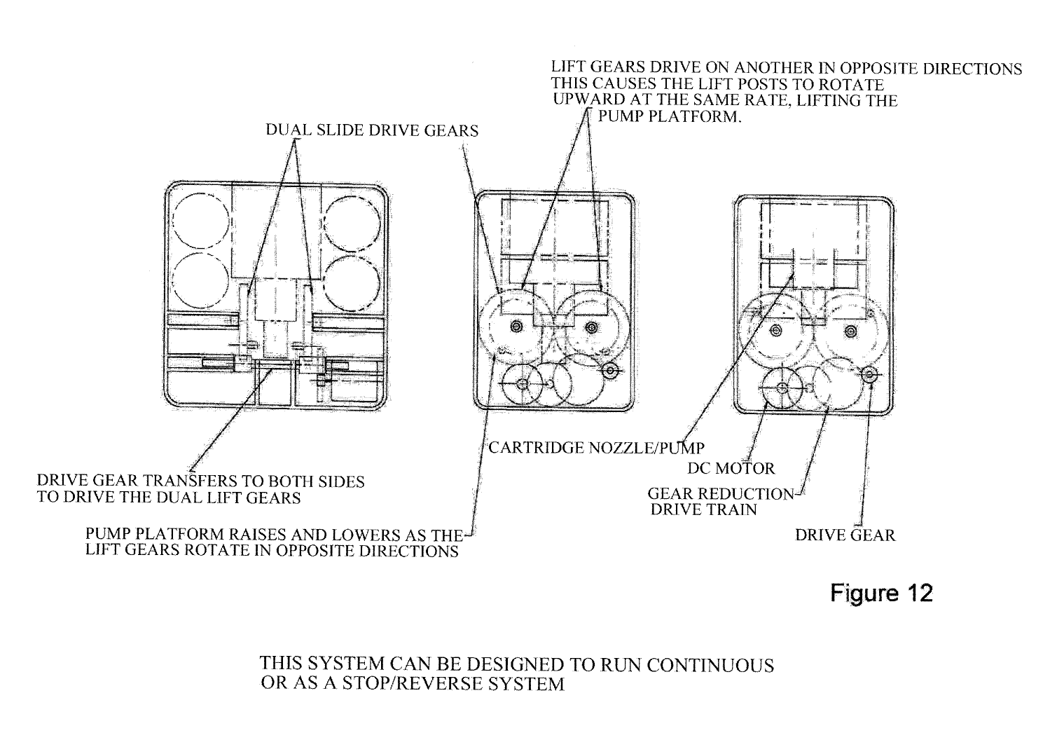

FIG. 12 shows an automotive direct drive gear platform mechanism with a DC motor. This system can run continuously or as a stop/reverse system.

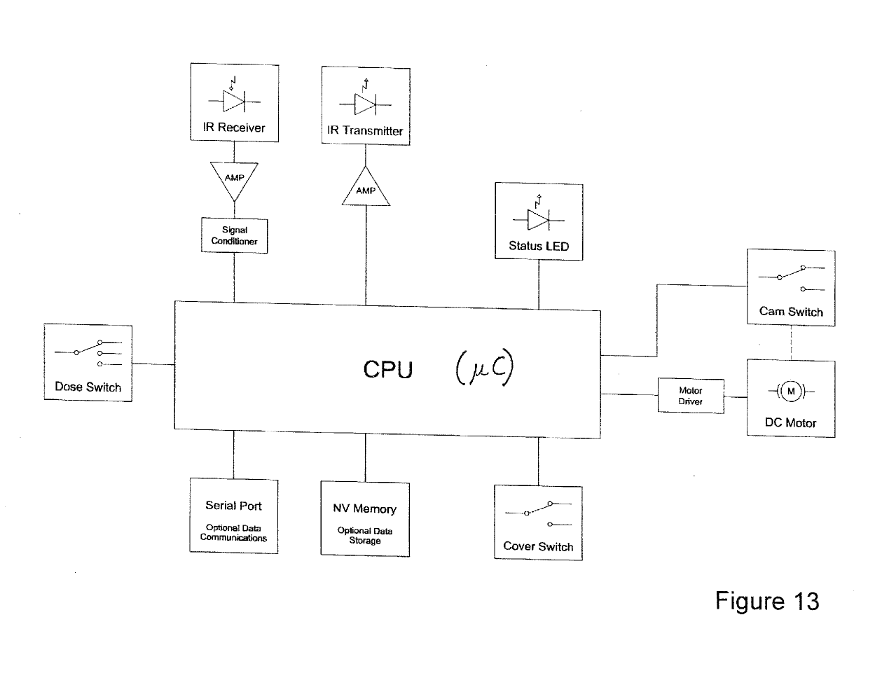

FIG. 13 is a block diagram of control circuit, which includes a microcontroller (μC). Connected to the microcontroller is a dose switch having several settings. According to the setting, the microcontroller can control the amount of time that the motor driver will drive the D.C. motor. A cam switch is shown which closes when a cam on the motor shaft depresses the switch. The cam/cam switch can be replaced by an optical sensor or magnetic sensor.

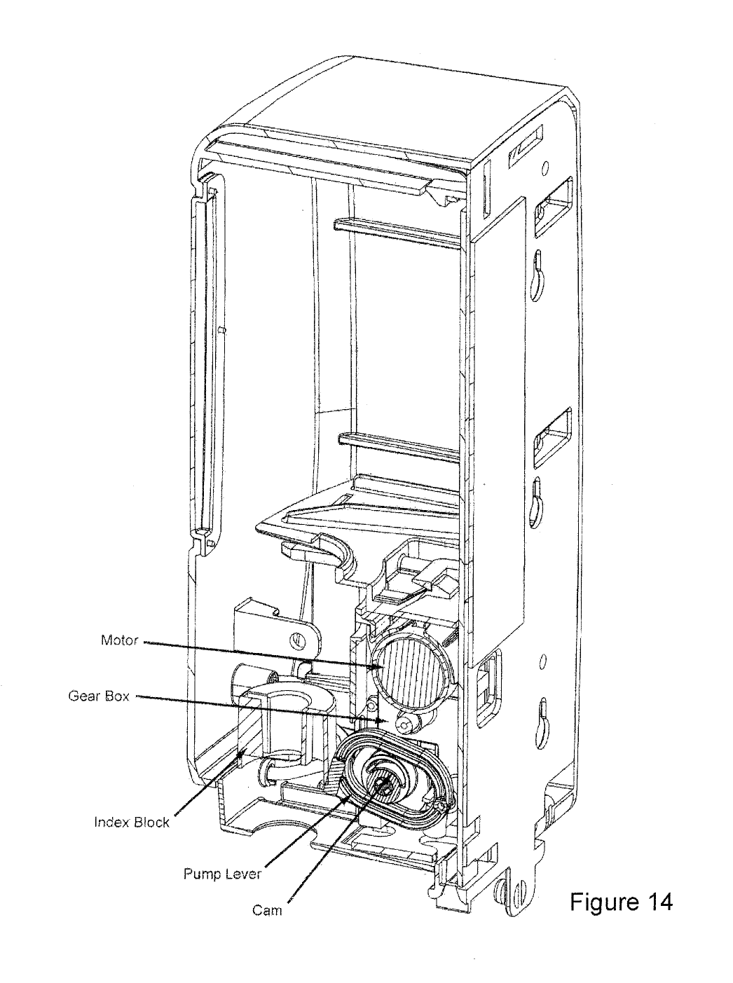

FIG. 14 is a cross-section view of a dispenser showing a motor, gear box and cam driven by the motor through the gear box. The cam will cause the pump lever to pivot about a pivot point on the right, and cause the index block to go up and down for each complete revolution of the cam. This will cause dispensing of a unit dose. Additional revolution(s) of the cam will increase the dose dispensed. If less than a unit dose is required, for example one-half of a unit dose, the cam can be controlled to rotate to raise the index block about halfway, then stop, then reverse to lower the index block back to its start lowest position. This will effectively cause dispensing of about one-half a unit dose. Cam switches can be arranged and positioned to detect the position of the pump lever, and location (height) of the index block to provide sensing and feedback to the microcontroller, so that the microcontroller knows the position of the index block, and thus the amount of time that the valve is open to dispense product. For example, a switch can be located about halfway up the vertical travel of the index block, so that when the index block reaches vertically halfway, the switch closes, the microcontroller detects this, and reverses the motor, to dispense only half a unit dose.

The systems with DC motors are automatic-type or touch-free systems, wherein a sensor or the like detects the presence of a user and energizes the motor. The DC motor systems can also be activated by a manual switch, such as by a foot, arm, or other body part, to minimize cross-contamination, or even by a user's hand.

Several embodiments have been shown and described, but the invention is not limited to these embodiments, and is defined only by way of the following claims.

What is claimed is:

1. A dispenser for dispensing sanitizer solution for hand hygiene, comprising:
a valve having an open position for dispensing sanitizer solution, and a closed position for preventing dispensing of sanitizer solution;
a sensor for sensing the presence of a user's hands; and
a control circuit responsive to the sensor for controlling the time that the valve is open and thus control the amount of solution dispensed.

2. The dispenser according to claim 1, further including a timer for setting the amount of time, wherein the timer is adjustable.

3. The dispenser according to claim 2, wherein the control circuit comprises a microcontroller, a motor controlled by the microcontroller, said motor controlling the time that the valve is open.

4. The dispenser according to claim 3, further including a cam driven by the motor which drives a lever arm which opens the valve.

5. The dispenser according to claim 1, further including a circumferential cam having a plurality of ribs arranged circumferentially around the cam, wherein rotation of the cam causes the ribs to sequentially engage a switch, the number of ribs engaging the switch determining the amount of time that the valve is open and the amount of solution dispersed.

6. The dispenser according to claim 1, wherein the control circuit includes:
a cam driven by a motor which controls opening of the valve; by
an optical transmitter and optical sensor, the cam defining openings through which tight from the optical transmitter passes to reach the optical sensor upon rotation of the cam, the optical sensor activating the opening of the valve when light is received from the optical transmitter through the openings.

7. The dispenser according to claim 1, wherein the control circuit includes:
a cam driven by a motor which controls opening of the valve by at least one magnet mounted on the cam, and at least one magnetic sensor which detects the magnet when the cam rotates, the magnetic sensor activating the opening of the valve when the magnetic sensor detects the magnet.

8. The dispenser according to claim 1, wherein the control circuit comprises a stepper motor which opens the valve only when the motor is stepped.

9. The dispenser according to claim 8, wherein the stepper motor is stepped a plurality of cycle times for each dispensing.

10. The dispenser according to claim 1, wherein the control circuit includes a D.C. motor for controlling the time that the valve is open, said motor being energized by a D.C. source and a step-up converter which outputs a D.C. voltage greater than the voltage of the D.C. source.

11. The dispenser according to claim 10, wherein the D.C. source comprises batteries connected in parallel.

12. A dispenser for dispensing sanitizer solution for hand hygiene, comprising:
a valve having an open position for dispersing sanitizer solution, and a closed position for preventing dispensing of sanitizer solution, said valve comprising a dose adjustable index block, said index block having a ramp with a plurality of ramp seats at different axial positions along the index block, and a dose ramp pin which can be moved between the ramp seats, and wherein the amount of sanitizer solution dispensed is dependent on the ramp seat in which the ramp pin is seated.

13. A dispenser for dispersing sanitizer solution for hand hygiene comprising:
a valve having an open position for dispensing sanitizer solution, and a closed position for preventing dispensing of sanitizer solution, said valve comprising a piston slideable within a cylinder to the open and closed positions; and
a push bar for actuation by a user, said push bar pivoting on a pivot point, said push bar raising a helix activation mechanism which slides the piston to open the valve in response, to dispense sanitizer solution.

14. The dispenser according to claim 13, wherein the cylinder has a valve seat at one end, and the piston rests on the valve seat to close the valve, and wherein the valve is open when the piston is displaced from the valve seat.

15. A dispenser for dispensing sanitization solution for hand hygiene, comprising:
a valve having an open position for dispensing sanitizer solution, and a closed position for preventing dispensing of sanitizer solution, said valve comprising a piston slideable within a cylinder to the open and closed positions;
a sensor for sensing the presence of a user's hands;
a motor for opening the valve; and
a motor controller for energizing the motor to raise an activation mechanism to open the valve in response to the sensor sensing a user's hands.

16. The dispenser according to claim 15, wherein the cylinder has a valve seat at one end, and the piston rests on the valve seat to close the valve, and wherein the valve is open when the piston is displaced from the valve seat.
